(19) United States
(12) Patent Application Publication
(10) Pub. No.: US 2017/0312202 A1
(43) Pub. Date: Nov. 2, 2017

(54) Synergistic Cationic Surfactant System

(71) Applicant: Colgate-Palmolive Company, New York, NY (US)

(72) Inventors: Elisa Navarro Flores, Mexico City (MX); Ramon Arnoldo LOPEZ BACA, Mexico City D.F. (MX); Ana ZULETA HERNANDEZ, Mexico City D.F. (MX); Victor GALICIA, Mexico City D.F. (MX)

(73) Assignee: Colgate-Palmolive Company, New York, NY (US)

(21) Appl. No.: 15/520,186

(22) PCT Filed: Oct. 23, 2014
(86) PCT No.: PCT/US2014/062068

§ 371 Date: Apr. 19, 2017

(51) Int. Cl. A61K 8/41; A61Q 5/12

(52) U.S. Cl. CPC A61K 8/416; A61Q 5/12

(57) ABSTRACT

The present disclosure pertains to compositions that include the synergistic combination of cationic surfactants cetyl trimethyl ammonium chloride and quaternium-82, methods involving the application of such compounds to hair, methods for making hair conditioners using such compounds, and uses of compositions containing such compounds for conditioning of hair for reducing tangling, increasing the ease of combing, increasing softness, and increasing luster/shine.

### BACKGROUND

There are a wide variety of consumer products for cleansing and conditioning of hair. Hair conditioners include leave-on and rinse-off products. Shampoos may consist of dedicated products that are solely for cleansing, or may include hair conditioning agents that permit a single product to both cleanse and condition the hair. Other products that are not intended solely as cleansing or conditioning compositions may include conditioning agents. The active components of conditioning products may be included to enhance the softness, manageability, luster, or moisture perception of hair, to reduce dryness, tangling, brittleness, or color loss, or to provide any combination of these benefits. While hair conditioners initially consisted of naturally-occurring oils, modern hair care products can make use of silicone, fatty alcohols, quaternary ammonium compounds, and other actives in order to accomplish a conditioning effect.

Many existing conditioning products may be effective to provide one or several particular benefits, but are unable to confer another desirable effect. Accordingly, there exists an ongoing need for conditioning agents and products that are made from such agents that provide any of the traditionally desired benefits of hair conditioning.

### BRIEF SUMMARY

The present disclosure concerns methods comprising applying to the hair of a mammal a composition that applying to the hair of a mammal a composition that includes 0.03% to 0.9% by weight cetyl trimethyl ammonium chloride and 0.2% to 1.9% by weight quaternium-82.

Also disclosed are compositions for the conditioning of hair, methods for making a hair conditioner, and uses of a composition for conditioning of hair, each of which involve the above described composition.

Further areas of applicability of the present invention will become apparent from the detailed description provided hereinafter. It should be understood that the detailed description and specific examples, while indicating the preferred embodiment of the invention, are intended for purposes of illustration only and are not intended to limit the scope of the invention.

### DETAILED DESCRIPTION

The following description of the preferred embodiment(s) is merely exemplary in nature and is in no way intended to limit the invention, its application, or uses.

As used throughout, ranges are used as shorthand for describing each and every value that is within the range. Any value within the range can be selected as the terminus of the range. In addition, all references cited herein are hereby incorporated by referenced in their entireties. In the event of a conflict in a definition in the present disclosure and that of a cited reference, the present disclosure controls.

Unless otherwise specified, all percentages and amounts expressed herein and elsewhere in the specification should be understood to refer to percentages by weight. The amounts given are based on the active weight of the material.

Provided herein are methods comprising applying to the hair of a mammal a composition that includes 0.03% to 0.9% by weight cetyl trimethyl ammonium chloride and 0.2% to 1.9% by weight quaternium-82. It has presently been discovered that the combination of these components in the specified amounts produces synergistic effects that include reducing the forces necessary for combing of hair and detangling of hair, increasing softness, and increasing shine/luster. Furthermore, the respective levels of cetyl trimethyl ammonium chloride and quaternium-82 components can be adjusted within the specified ranges in order to provide medium, high, and highest levels of conditioning, as desired.

In some embodiments, the compositions according to the present methods may include 0.03% to 0.3% cetyl trimethyl ammonium chloride. In other embodiments, the compositions may include 0.08% to 0.2% by weight cetyl trimethyl ammonium chloride. For example, the compositions may include about 0.03%, 0.04%, 0.05%, 0.06%, 0.07%, 0.08%, 0.09%, 0.1%, 0.15%, 0.2%, 0.25%, 0.3%, 0.35%, 0.4%, 0.45%, 0.5%, 0.55%, 0.6%, 0.65%, 0.7%, 0.75%, 0.8%, 0.85%, or 0.9% by weight cetyl trimethyl ammonium chloride.

The compositions of the present invention also include quaternium-82. Quaternium-82 is quaternary ammonium salt prepared by reacting oleic acid with N,N,N₁,N₁-tetrakis (2-hydroxypropyl) ethylene-diamine, followed by quaternization with dimethyl sulfate. It possesses the following structure:

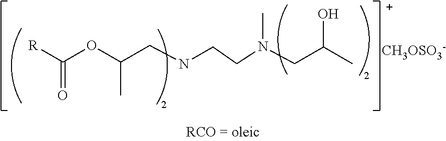

Stepanquat® ML, which is available from Stepan Company (Northfield, Ill.), represents an exemplary source of quaternium-82.

The compositions also include 0.2% to 1.9% by weight quaternium-82. In some embodiments, the compositions include 0.8% to 1.9% by weight quaternium-82. In other embodiments, the compositions include 0.9% to 1.6% by weight quaternium-82. For example, the compositions may include about 0.2%, 0.25%, 0.3%, 0.35%, 0.4%, 0.45%, 0.5%, 0.55%, 0.6%, 0.65%, 0.7%, 0.75%, 0.8%, 0.85%, 0.9%, 0.95%, 1.0%, 1.1%, 1.2%, 1.3%, 1.4%, 1.5%, 1.6%, 1.7%, 1.8%, or 1.9% by weight quaternium-82.

In certain embodiments, the compositions include about 0.50% by weight cetyl trimethyl ammonium chloride and about 0.50% by weight quaternium-82, about 0.75% by weight cetyl trimethyl ammonium chloride and about 0.25% by weight quaternium-82, or about 0.25% by weight cetyl trimethyl ammonium chloride and about 0.75% by weight quaternium-82.

The compositions of the present invention may further comprise one or more of a thickener, a viscosity enhancer, a pH modifier, a further surfactant, an emulsifier, an oil, a detangling agent, a moisturizer, a hair shine enhancer, a hair strength enhancer, a preservative, and fragrance. For example, the compositions may comprise each of a thickener, a viscosity enhancer, a pH modifier, a further surfactant, an emulsifier, an oil, a detangling agent, a moisturizer, a hair shine enhancer, a hair strength enhancer, a preservative, and fragrance. In a certain embodiment, the composition comprises water, hydrophobically modified hydroxyethylcellulose, cetyl stearyl alcohol, glyceryl monostearate, mineral oil, a cationic silicone emulsion, a polyalkylsiloxane emulsion, honey, hydrolyzed silk, a preservative, fragrance, NaOH, and citric acid. The compositions may take the form of a hair conditioner, such as a rinse-off hair conditioner.

The compositions of the present invention may comprise fatty alcohols, such as stearyl alcohol, cetyl alcohol, or both. For example, the compositions may include cetostearyl alcohol. The compositions may also or alternatively include silicones, such as dimethicone, dimethiconol, cyclopentasiloxane, an amodimethicone, or any combination thereof. The compositions may also or alternatively include additional cationics, including surfactants like cetrimonium chloride, or polymeric cationics, such as the polyquaternium series (e.g., polyquaternium-6, -7, or -8) or cationic guar gums. In certain embodiments, the compositions do not include any cationic surfactants other than the cetyl trimethyl ammonium chloride and quaternium-82. The compositions may further comprise oils, such as mineral oil. Further examples of fatty alcohols, silicones, cationics, and oils are well known among those skilled in formulating hair products.

The presently disclosed methods may further comprise detangling the hair after application of the composition. Detangling may be performed mechanically, such as with a comb or brush, or manually. Detangling may involve the application of a various peak load forces. The present methods accord with the unexpected discovery that when a composition of the type described above is applied to hair, the peak load forces that are required to detangle the hair are lower than those that are required to detangle a comparable portion of hair to which the present compositions have not been applied, and are even lower than the forces that are required after application of alternative compositions, such as hair conditioners, that do not include the specified compositions. Furthermore, the peak load forces are less than are required after application of a composition that includes different amounts of CTAC and quaternium-82. This feature of the present methods and others are discussed more fully infra. The peak load force that is required to detangle a tress of hair weighing about 3.5 g after application of a composition according to the present claims may be, for example, no more than about 400 gmf, no more than about 375 gmf, no more than about 350 gmf, no more than about 325 gmf, no more than about 300 gmf, no more than about 275 gmf, no more than about 250 gmf, or no more than about 240 gmf. Hair tress is about 20-22 cm long.

The present methods may further comprise combing the hair after application of the composition. “Combing” may be performed mechanically, such as with a comb or brush, or manually. Combing may involve the application of a various amounts of force (work) as expressed in joules. The presently disclosed methods accord with the unexpected discovery that when a composition of the type described above is applied to hair, the work that is required to comb the hair is lower than that which is required to comb a comparable portion of hair to which the present compositions have not been applied, and is even lower than the work that is required after application of alternative compositions, such as hair conditioners, that do not include the specified compositions. Furthermore, the work is less than are required after application of a composition that includes different amounts of CTAC and quaternium-82. This feature of the present methods and others are discussed more fully infra. The work that is required to comb a tress of hair weighing about 3.5 g after application of a composition according to the present claims may be, for example, no more than about 0.5 joules, no more than about 0.48 joules, no more than about 0.45 joules, no more than about 0.43 joules, no more than about 0.4 joules, no more than about 0.38 joules, or no more than about 0.36 joules.

The present disclosure also pertains to compositions for the conditioning of hair comprising 0.03% to 0.9% by weight cetyl trimethyl ammonium chloride and 0.2% to 1.9% by weight quaternium-82. For example, the present compositions may be a hair conditioner, such as a rinse-off hair conditioner.

Unless otherwise specified, any component, element, attribute, or step that is disclosed with respect to one embodiment of the present methods, compositions, and uses may apply to any other embodiment that is disclosed herein. For example, the presently disclosed compositions may have any of the characteristics of the composition that are used pursuant to the present methods, and vice versa, and the compounds that are used in the presently disclosed methods may have any of the characteristics of the compounds of the presently disclosed compositions, and vice versa.

Thus, the CTAC and quaternium-82 in the present compositions may have any of the characteristics of the compounds described above with respect to the disclosed methods that involve the use of these components. In addition, the present compositions may have any of the characteristics of the compositions that are used pursuant to the above-described methods.

The presently disclosed compositions may also include one or more further ingredients that are suitable for inclusion in the composition for use on the hair of a mammal. As used herein, one or more further ingredients that are suitable for inclusion in the composition for use on the hair of a mammal refers to any ingredient that is characteristic of a hair product, such as a hair conditioner. Exemplary ingredients that are characteristic of a hair product include fatty alcohols, silicones, oils, and additional cationics. Examples of these ingredients are described above in connection with the present methods, and further examples of which are well known among those skilled in formulating hair products. The component that is characteristic of a hair product may be, but is not necessarily, an ingredient that is not typically included in a non-hair product, such as in a fabric conditioner. The present compositions may also include components that are not necessarily characteristic of a hair product.

The present disclosure also pertains to methods for making a hair conditioner comprising preparing a composition as previously described (i.e., comprising 0.03% to 0.9% by weight cetyl trimethyl ammonium chloride and 0.2% to 1.9% by weight quaternium-82), and one or more further ingredients that are suitable for inclusion in the composition for use on the hair of a mammal. The compositions may have any of the characteristics described above with respect to the above described methods that involve the use of CTAC and quaternium-82. In addition, the compositions may have any of the characteristics that are associated with the above-described methods. Likewise, the one or more further ingredients that are suitable for inclusion in the composition for use on the hair of a mammal may have any of the characteristics that are described in connection with the above-described compositions, such as in the immediately preceding paragraph.

Also disclosed herein are uses of a composition for conditioning of hair for one or both of reducing tangling of the hair and increasing ease of combing of the hair, wherein the composition comprises 0.03% to 0.9% by weight cetyl trimethyl ammonium chloride and 0.2% to 1.9% by weight quaternium-82, as previously described. For example, the composition may be a hair conditioner, such as a rinse-off hair conditioner. The composition may further include one or more further ingredients that are suitable for inclusion in the composition for use on the hair of a mammal. The composition may have any of the characteristics described above with respect to the above described methods that involve the use of the compositions. In addition, the compositions may have any of the characteristics of the compositions that are associated with the above-described methods. Likewise, the one or more further ingredients that are suitable for inclusion in the composition for use on the hair of a mammal may have any of the characteristics that are described in connection with the above-described compositions.

### EXAMPLES

### Example 1

Clean, bleached hair was prepared into 3.5±0.1 g tresses and pretreated with a cleansing solution of 20% ammonium lauryl sulfate then dried overnight.

Hair tresses were wet under tap water at 25° C. and then treated with 0.5 mL of rinse-off conditioner prototypes respectively having different quantities of cetyl trimethyl ammonium chloride and quaternium-82 (see Table 1, below). Quaternium-82 was provided in the form of Stepanquat® ML (Stepan Company, Northfield, Ill.). Hair was rubbed for one minute with the test conditioner, and then rinsed again under tap water for one further minute. A Dia-Stron instrument (Broomall, Pa.) was used to determine the peak load force (gmf), which indicates the force required to untangle a hair tress. The instrument also measured the total work force (joules) as the force necessary to comb a hair tress.

Table 1, below, lists the amounts of active components cetyl trimethyl ammonium chloride (CTAC) and quaternium-82 in the prototype conditioner formulas used:

TABLE 1

| | CTAC | Quaternium-82 |
|---|---|---|
| Sample | (weight %) | (weight %) |
| 1 (comparative) | 1 | 0 |
| 2 (comparative) | 0 | 1.5 |
| 3 | 0.5 | 0.5 |
| 4 | 0.75 | 0.25 |
| 5 | 0.25 | 0.75 |
| 6 (comparative) | 0 | 2.00 |
| 7 (comparative) | 0.75 | 0.10 |

Sample 1, 2 and 6 represent conventional formulations in which only one of cetyl trimethyl ammonium chloride (CTAC) and quaternium-82 are present.

Five separate tresses were measured for each composition under test. Each tress was measured three times, for a total of 15 readings for each test composition.

Table 2 shows the average force (gmf) required to detangle a hair tress that was treated by one of the seven test formulations:

TABLE 2

| Sample | average force (gmf) |
|---|---|
| 1 (comparative) | 555 |
| 2 (comparative) | 367 |
| 3 | 247 |
| 4 | 300 |
| 5 | 234 |
| 6 (comparative) | 414 |
| 7 (comparative) | 511 |

Table 3 shows the average force (joules) required to comb a hair tress that was treated by one of the seven test formulations:

TABLE 3

| Sample | average force (joules) |
|---|---|
| 1 (comparative) | 0.87 |
| 2 (comparative) | 0.51 |
| 3 | 0.37 |
| 4 | 0.45 |
| 5 | 0.35 |
| 6 (comparative) | 0.66 |
| 7 (comparative) | 0.8 |

Test compositions 3, 4, and 5 successfully reduced the force necessary to untangle hair, and the force necessary to comb hair, relative to conventional compositions 1, 2, and 6.

What is claimed is:

1. A method for detangling hair of a mammal, comprising:
applying to the hair of the mammal a composition that includes 0.03% to 0.9% by weight cetyl trimethyl ammonium chloride and 0.2% to 1.9% by weight quaternium-82.

2. The method of claim 1, wherein the composition comprises 0.03% to 0.3% by weight cetyl trimethyl ammonium chloride.

3. The method of claim 2, wherein the composition includes 0.08% to 0.2% by weight cetyl trimethyl ammonium chloride.

4. The method of claim 3, wherein the composition includes about 0.1% by weight cetyl trimethyl ammonium chloride.

5. The method of claim 1, wherein the composition includes 0.8% to 1.9% by weight quaternium-82.

6. The method of claim 5 wherein the composition includes 0.9% to 1.6% by weight quaternium-82.

7. The method of claim 6, wherein the composition includes about 1% by weight quaternium-82.

8. The method of claim 1, further comprising detangling the hair.

9. The method of claim 8 wherein the peak load force required to detangle a tress of the hair weighing 3.5+/−0.1 g is no more than 400 gmf.

10. The method of claim 1, further comprising combing the hair.

11. The method of claim 10 wherein the work required to comb a tress of the hair weighing 3.5 g+/−0.1 is no more than 0.5 joules.

12. A composition for conditioning hair, comprising:
0.03% to 0.9% by weight cetyl trimethyl ammonium chloride; and
0.2% to 1.9% by weight quaternium-82.

13. The composition of claim 12, wherein the composition comprises 0.03% to 0.3% cetyl trimethyl ammonium chloride.

14. The composition of claim 13, wherein the composition comprises 0.08% to 0.2% by weight cetyl trimethyl ammonium chloride.

15. The composition of claim 14, wherein the composition comprises about 0.1% by weight cetyl trimethyl ammonium chloride.

16. The composition of claim 12, wherein the composition comprises 0.8% to 1.9% by weight quaternium-82.

17. The composition of claim 16, wherein the composition comprises 0.9% to 1.6% by weight quaternium-82.

18. The composition of claim 17, wherein the composition comprises about 1% by weight quaternium-82.

19. (canceled)

20. The composition of claim 12, wherein the composition is a rinse-off hair conditioner.

21. A method for making a hair conditioner comprising mixing the composition of claim 12 with one or more further ingredients suitable for inclusion in the composition for use on hair of a mammal.

22. (canceled)
